(19) United States
(12) Patent Application Publication
(10) Pub. No.: US 2009/0304681 A1
(43) Pub. Date: Dec. 10, 2009

(54) Methods for the treatment and prevention of infection using anti-selectin agents

(76) Inventors: Mark A. Jutila, Bozeman, MT (US); Robert F. Bargatze, Bozeman, MT (US); Aiyappa Palecanda, Mysore (IN); Pati M. Giee, Bozeman, MT (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/658,748

(22) PCT Filed: Jul. 27, 2005
(86) PCT No.: PCT/US05/26529

§ 371 Date: Jun. 15, 2009

Related U.S. Application Data
(60) Provisional application No. 60/591,925, filed on Jul. 29, 2004.

(51) Int. Cl. A61K 39/395; A61P 11/06; A61P 11/00

(52) U.S. Cl. 424/133.1; 424/137.1

(57) ABSTRACT

Methods for the treatment and prevention of pulmonary infections are disclosed, in particular, methods comprising the administration of one or more anti-selectin agents to a patient diagnosed with a pulmonary infection. Anti-selectin agents that inhibit leukocyte recruitment to the lungs have been found to be beneficial for the treatment of lung infections, including infections associated with chronic bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis, sinusitis and pulmonary fibrosis.

### STATEMENT OF GOVERNMENT RIGHTS

This invention was made in part with support from the National Institutes of Health (Grant No. AI43789). The Government may have certain rights in the invention.

### FIELD OF THE INVENTION

The present invention provides methods for the treatment and prevention of infections, particularly pulmonary infections, by the administration of anti-selectin agents.

### BACKGROUND

Following respiratory infection, airway epithelial cells produce a range of cytokines, including chemokines, which promote recruitment and activation of inflammatory cell types including T-cells, natural killer (NK) cells, macrophages, eosinophils and neutrophils. Message and Johnston, 2004, J. Leuk. Biol. 75: 5-17. For instance, cystic fibrosis lung disease is characterized by an excessive inflammatory response, often associated with chronic Pseudomonas aeruginosa infection. Bronchoalveolar lavage in healthy subjects and subjects with cystic fibrosis lung disease showed that subjects with cystic fibrosis have elevated proinflammatory cytokines but negligible amounts of the anti-inflammatory cytokine interleukin-10. Chmiel, et al., 1999, Am. J. Respir. Crit. Care Med. 160: 2040-2047. Other pro-inflammatory cytokines have also been implicated in the pathology of pulmonary infections. For example, interleukin-1 causes neutrophil infiltration and elevated levels of IL-1 have been found in pleural fluids of patients with emphysema. Sila-Mejias, et al., 1995, Chest 108: 942-945. Inflammatory cell infiltration from the blood is regulated by various adhesion molecules expressed on the cell surface of leukocytes, endothelial cells and platelets.

A family of cell surface glycoproteins, the “selectins,” has been identified in some instances as mediators of leukocyte recruitment and migration to regions of inflammation. Lasky, 1992, Science 258 (5084):964-9. For example, selectins have been implicated in the inflammatory process of disease states such as chronic obstructive pulmonary disease (COPD) (Noguera, et al., 2004, Chest, 125(5):1837-42), asthma (Sjosward, et al., 2004, Respiration 71(3): 241-5), reactive arthritis, rheumatoid arthritis (RA) and sepsis (Kuuliala, et al., 2004, Scand. J. Rheumatol. 33(1): 13-8) as well as in inflammatory processes in the skin, lungs and gut (Ley and Kansas, 2004, Nat. Rev. Immunol. 4(5):325-36). Further it has been shown that inflammation is decreased via anti-adhesion blocking, including anti-selectin blocking (See, U.S. Pat. No. 6,030,947). Pro-inflammatory cytokines have also been shown to be beneficial for the treatment of infections. (See generally, Dube, et al., 2004, Infect. Immun. 72(6):93561-70. Ribeiro, et al., 2004, Infect. Immun. 72(6):3391-7, Koedel., et al., 2004, Brain April 28 [Epub ahead of print]). For example, IL-1 has also been implicated in the containment of infections by Listeria monocytogenes and Leishmania major and Myocobacterium tuberculosis. See, for example, Juffermans, et al., 2000, J. Infect. Dis. 182(3):902-8.

Leukocyte recruitment is also beneficial in helping to eradicate infection. For instance, it is hypothesized that recruitment of leukocytes to the site of a wound or infection helps ameliorate or eradicate a wound or infection by restricting the extent of the infection. (U.S. Pat. No. 6,713,605, Message and Johnston, 2004, J. Leuk. Biol. 75: 5-17). Therefore although anti-inflammatory treatment and anti-adhesion blocking have both been shown to be beneficial to reducing inflammation it would be counter intuitive to use such treatments for the eradication of an infection itself, given the prior conclusion amongst experts in the field that pro-inflammatory activities are beneficial for treating infections rather than anti-inflammatory activities.

Pulmonary infection can be lethal. From 1979 to 1994, the overall crude death rates for pneumonia and influenza in the United States increased 59%, from 20.0 to 31.8 deaths per 100,000 population. From 1979 to 1992, the pneumonia and influenza death rate, age-adjusted to a 1980 standard population, increased 22%, from 20.4 to 24.8. Centers for Disease Control, Morbidity and Mortality Weakly Report, Jul. 21, 1995. The incidence of tuberculosis, pulmonary fungal infections and atypical pulmonary infections has risen since steadily since the emergence of AIDS nearly 20 years ago. New methods of treating pulmonary infections are needed.

### SUMMARY

The present inventors have surprisingly found that anti-selectin agents that inhibit leukocyte recruitment are useful for the treatment and prevention of infectious disease, especially pulmonary infections. The present invention provides methods of treating or preventing a pulmonary infection comprising administering to a patient in need thereof a therapeutically effective amount of an anti-selectin agent, wherein said treatment reduces pathogen load in the lung.

In one embodiment, the invention provides methods for treating a pulmonary infection wherein the anti-selectin agent is a competitive binding agent that binds to E-selectin, L-selectin or P-selectin or combinations thereof. In one embodiment, the anti-selectin agent is an antibody or antigen binding fragment thereof that specifically binds to an antigenic determinant on E-selectin, P-selectin, L-selectin or combinations thereof. In a preferred embodiment, the anti-selectin agent is a monoclonal antibody that is effective in a dose range of 0.05 mg/kg to 5 mg/kg. More preferably, the anti-selectin agent is a monoclonal antibody effective at a concentration of about 1 mg/kg. In a preferred embodiment, the anti-selectin agent is the monoclonal antibody, EL246

In certain embodiments, the invention provides methods of treating pulmonary infections caused by a pathogen. In certain embodiments, the invention provides methods of treating pulmonary infections caused by bacteria, virus, fungi or combinations thereof. In a preferred embodiment, the invention provides methods for the treatment and prevention of bacterial pulmonary infections caused by Pseudomonas aeruginosa. In a more preferred embodiment, the invention provides methods for preventing or reducing the frequency or severity of acute exacerbations of pulmonary diseases.

In certain embodiments, the invention provides methods of treating pulmonary infections in a subject. In a preferred embodiment, the subject is a mammal. Mammals that would benefit from the methods of the invention include agricultural animals such as cows, horses, sheep and pigs and domestic animals and pets such as dogs and cats and exotic animals housed in zoological exhibits. In a more preferred embodiment, the subject is a human.

In certain embodiments, the invention provides methods for treating a pulmonary infection in a subject, wherein the subject is afflicted with chronic bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis, sinusitis and pulmonary fibrosis. The present invention is also useful in anti-terrorism platforms for the treatment and prevention of highly contagious and virulent pulmonary infections.

### BRIEF DESCRIPTION OF THE FIGURES

FIG. 1 provides a graph illustrating the effect of monoclonal antibody EL246 on the clearance of Pseudomonas aeruginosa from ovine lung;

FIG. 2 provides a graph illustrating the effect of EL246 on the clearance of non-Pseudomonas bacteria from ovine lung;

FIG. 3 provides seven graphs illustrating the effect of EL246 on total cells, neutrophils, macrophages, interleukin-8 (IL-8), myeloperoxidase, protein and interleukin-1beta in monkeys following LPS induced inflammation;

FIG. 4 provides a schematic of the ProteoFlow® shear assay system;

FIG. 5 provides a graph illustrating the rolling interaction of various doses of EL246 in the ProteoFlow® assay;

FIG. 6 provides a graph illustrating the average percent inhibition of U937 cells on recombinant E-selectin as a function of dose of EL246;

FIG. 7 provides a graph illustrating the average percent inhibition of U937 cells on CHO E-selectin as a function of dose of EL246; and

FIG. 8 provides a graph illustrating the percent inhibition of U937 cells on activated HUVECs as a function of dose of EL246.

### DETAILED DESCRIPTION OF THE INVENTION

Definitions. The following terms shall have the meaning as described herein.

The term “pulmonary” means in any part of the lung tissue, particularly in the respiratory epithelium.

The term “infection” means symptomatic colonization of at least one pathogen. Symptomatic colonization can be manifest as an objective or subjective indicator of infection including fever, cough, sputum production, a change in the color of sputum (usually from a clear to green color), increased viscosity of the sputum, increased leukocyte count, dyspnea, malaise, tightness in the chest, hypoxia and/or pleuritic chest pain. Pulmonary infection can be evaluated by objective indicators of gas exchange and oxygenation of the blood as measured by, for example, pulse oximetry and arterial blood gases. Pulmonary infection can also be evaluated by the presence and number of indicators recovered during bronchoalveolar lavage (BAL). Indicators can be cells, cytokines or proteins recovered using BAL from a subject afflicted with or suspected of being afflicted with a pulmonary infection. Indicators can be, for example, neutrophils, interleukin-8, interleukin 1-beta, myeloperoxidase, protein and total cell count.

The term “symptomatic” is an indication of disease or injury, whether observed or noted by the infected subject or by another, usually a health care provider or caregiver.

The term “colonization” is the establishment of the pathogen in the pulmonary tissue.

The terms “treat,” “treats,” “treating” and “treatment” are procedures or applications that are intended to eradicate, ameliorate or relieve infection.

The term “reduce pathogen load in the lung” or “reduces pathogen load in the lung” means a relative reduction in the number of pathogenic organisms (bacteria, virus or fungi) in the subject's respiratory tract that cause symptoms in the colonized subject. The relative reduction in the number of pathogenic organisms means a reduction in pathogen load in an afflicted subject following treatment as compared to pathogen load prior to treatment.

The term “IC50” means the inhibitory concentration at which growth or activity is inhibited by fifty percent (50%). The term “EC50” means the effective concentration that provokes a response halfway between the baseline and maximum response.

The terms “acute exacerbation” and “acute exacerbations” mean a sudden increase in the severity of pulmonary disease. An acute exacerbation typically manifests as worsening disease symptoms, significant reduction of lung function, increased coughing, dyspnea, sputum production or sputum purulence. It has been estimated that about 80% of acute exacerbations of COPD, for example, are of infectious origin (e.g. caused by bacteria or viruses) with non-infectious causes including environmental changes, pollution, non-compliance with medication regimens and allergic factors accounting for the other about 20%. Sethi, 2002, J. Resp. Dis. 23(4): 217-25.

The term “recruitment” means the step-fold process by which leukocytes interact with endothelial cells by rolling, signaling and sticking, which leads to migration through endothelial cells to the site of an infection. Leukocytes can be lymphocytes, macrophages, monocytes, neutrophils, eosinophils or combinations thereof.

The present invention provides methods of treating pulmonary infection in a subject comprising administering to a subject in need thereof a therapeutically effective amount of an anti-selectin agent, wherein said treatment reduces pathogen load in the lung. The present invention provides methods for improving lung physiology by augmenting the body's ability to clear infection with less or no concomitant anti-infective therapy. The present invention further provides methods for preventing or reducing the frequency or severity of acute exacerbations of pulmonary diseases.

The presence of infectious pathogenic organisms in a mammalian system triggers a number of host responses. The first line of defense comes from physical barriers such as the skin, mucus and cilia. This defense is generally followed by the release of a number of mediators that directly lyse pathogens, destroy pathogens through opsonisation and phagocytosis. The recruited inflammatory cells, through phagocytosis of opsonized and non-opsonized cells, facilitate clearance or destruction of the pathogen as well. The adaptive immune response includes the production of neutralizing antibodies and the responses of various immune cells including lymphocytes, macrophages, monocytes, neutrophils and eosinophils.

However, the inflammatory response is not altogether helpful. The influx of immune cells can also inflict tissue damage via the release of various cytokines, which induce more inflammation, and toxic compounds, such as reactive oxygen species. In the respiratory tract, this inflammatory process can be particularly destructive. The architecture of the lung allows gas exchange across the thin alveoli membranes as discussed in more detail below. The price of excessive or inappropriate inflammatory response can be decreased alveoli surface area and decreased gas exchange. The immune response to respiratory infection must, therefore, be rapid and efficient.

In many cases, the body will clear an infection after there is a decrease in inflammation and a return to normal cell physiology. For example, infection with the etiological cause of hantavirus causes a recruitment of neutrophils. Neutrophil influx damages the wall of the blood vessels allowing serum leakage into the pleural cavity and the fluid accumulation in the lung. Accepted therapy for hantavirus consists of supportive care while the body clears the infecting virus.

A similar pathology is at work during infections with Pneumocystis carinii. Upon infection with P. carinii, neutrophils are rapidly recruited and lung damage results due to fluid accumulation in the lung. Again, supportive care is administered while the body clears the infecting agent. However, in both of these examples supportive care must be aggressively pursued and frequently death still occurs.

Pulmonary Anatomy and Physiology

The lungs are responsible for gas exchange, their vital function dictating an elaborate architecture. The lungs are each enclosed within a double membrane known as the pleura. The right lung is the larger, being divided into three lobes, while the left is divided into two lobes. The lobes are further divided into bronchopulmonary segments, each of which has a segmental bronchus. The trachea branches off into the two main tubes of the lungs—the right and left bronchi. Within the lungs the bronchi branch again, forming secondary and tertiary bronchi, then smaller bronchioles, and finally terminal bronchioles. At the end of the terminal bronchioles are the alveoli. In all there are about 25 divisions between the trachea and the alveoli, with the structure of the tubes changing progressively from the trachea to the terminal bronchioles. (See, Merck Manual, 17th ed. Chapter 63, Textbook of Medical Physiology, 10th ed., Guyton and Hall, eds., 2000, Elsevier, Human Physiology, Silverthorn, 2000, Prentice-Hall, and Principles of Human Anatomy and Physiology, 10th ed. Tortora et al., eds. 2002, John Wiley and Sons.)

The upper respiratory tract has a wall comprising cartilage and smooth muscle, an epithelial lining with cilia and mucus-secreting goblet cells and endocrine cells. The lower respiratory tract has no cartilage, a progressively thinner muscular layer, a single layer of ciliated cells, few goblet cells and granulated Clara cells that produce a surfactant-like substance.

The alveolar sacs are made up of groups of alveoli at the end of the terminal bronchioles. Each lung contains approximately 300 million alveoli, giving a total surface area of about 40-80 m². The epithelial lining of the alveoli consists mainly of type I pneumocytes which provide a thin layer for gas exchange. They are connected to type II pneumocytes by tight junctions. These tight junctions limit the fluid movement in and out of the alveoli. Although more numerous than the type I pneumocytes, type II pneumocytes cover less epithelium. They contain vacuoles that produce the pulmonary surfactant. The alveoli also contain macrophages. (See, Merck Manual, 17th ed. Chapter 63, Textbook of Medical Physiology, 10th ed., Guyton and Hall, eds., 2000, Elsevier, Human Physiology, Silverthorn, 2000, Prentice-Hall, and Principles of Human Anatomy and Physiology, 10th ed. Tortora et al., eds. 2002, John Wiley and Sons, incorporated herein by reference in their entireties.)

Gas exchange occurs at the level of the bronchioles. The alveoli are served by a diffuse network of capillaries which provide a large surface area of approximately 30 m² for gaseous exchange. (See generally, U.S. Pat. No. 6,175,755, Labiris and Dolovich, 2003, Br. J. Clin. Pharmacol. 56(6): 588-99, Labiris and Dolovich, 2003, Br. J. Clin. Pharmacol. 56(6): 600-12.)

Oxygen from inhaled air passes through the alveoli into the bloodstream. The blood is then taken to the left side of the heart via the pulmonary veins, and from here it is pumped through the body. Deoxygenated blood, which returns from all areas of the body to the right side of the heart, is pumped back to the lungs via the pulmonary arteries. Carbon dioxide passes from the capillaries which surround the alveoli, into the alveolar spaces, and is breathed out.

The degree of oxygenation of a subject can be assessed both by visual observation and by objective measures. Visual observation can include color changes, especially in the extremities and nail beds. Objective measures can include oxygen saturation determinations by pulse oximetry or arterial blood gas levels. Both pulse oximetry and arterial blood gas (ABG) are measures of the partial atmospheric pressure of oxygen (PaO₂) in the blood. Pulse oximetry is a convenient indirect method of measuring PaO₂ and is commonly available. A resting individual without pulmonary disease will generally have a pulse oximeter reading of about 95-100%. A pulse oximeter indication of 97% correlates to a PaO₂ of about 97 mmHg. A pulse oximeter indication of 90% correlates to a PaO₂ of about 60 mmHg which is dangerously low. A pulse oximeter indication of 80% correlates to a PaO₂ of about 45 mmHg indicating severe hypoxia. (See, generally, Bierman, et al., 1992, Chest, 102(5): 1367-70, Gay, 2004, Resp. Care 49(1): 39-51, Davies et al., 2003 N. Z. Med. J. 116(1168): U297, Casey, 1997, Nurs. Stand. 15(47):46-53 and Middleton and Henry, 2000, Int. J. Clin. Pract. 54(7):438-44, each of which is incorporated herein by reference in its entirety).

In certain embodiments the treatment of a pulmonary infection can provide a pulse oximetry level of about ≧90%, about ≧93%, about ≧95%, about ≧97% in an infected subject. In certain embodiments the treatment of a pulmonary infection can provide a PaO₂ of about 60 mmHg, about 70 mmHg about 75 about 80 mmHg, about 85 mmHg, about 90 mmHg, about 95 mmHg or about 97 mmHg.

Bronchoalveolar lavage (BAL) is a diagnostic and therapeutic procedure conducted by placing a fiberoptic scope into the lung of a subject, and injecting sterile water (saline) into the lung and subsequently removing the water. The sterile water removed contains secretions, cells, and protein from the lower respiratory tract. Samples obtained by BAL can be analyzed to provide more information about possible disease processes going on in the lungs, as described in Example 1, below. Additionally, the complement of inflammatory and immune cells and inflammatory mediators that are identified can also be useful in assessing the disease state of the lung. (See generally, Sanchez Nieto, et al., 1995, Eur. J. Clin. Microbiol. Infect. Dis. 14(10):839-50, Arora, et al., 2002, Anaesth. Intensive Care 30(1): 11-20, Fiorini, et al., 2000, Biomed. Pharmacother. 54(5):274-8, Jarjour, et al., 2000, J. Allergy Clin. Immunol. 105: 1169-77 and Valles, et al., 1994, Eur. J. Clin. Microbiol. Infect. Dis. 13(7):549-58 each of which is incorporated herein by reference in its entirety.)

Spirometry can be used to monitor pulmonary function in conjunction with the methods of the present invention. Spirometry is a maneuver in which a subject inhales maximally from tidal respiration to total lung capacity (TLC) and then rapidly exhales to the fullest extent until no further volume is exhaled at residual volume (RV). The maneuver may be performed in a forceful manner to generate a forced vital capacity (FVC) measurement or in a relaxed manner to generate a slow vital capacity (SVC) measurement. In normal individuals, the inspiratory vital capacity, the expiratory SVC, and expiratory FVC are essentially equal. However, in patients with obstructive airways disease, the expiratory SVC is generally higher than the FVC. Values generated from a spirogram provide data regarding the mechanical properties of the lungs, including airflow (forced expiratory volume in 1 second, or FEV₁, along with other timed volumes) and exhaled lung volume (FVC or SVC). The measurements are generally expressed in liters for volumes or in liters per second for flow and can be corrected for body temperature and pressure of gas that is saturated with water vapor. (See, Pulmonary Function Testing: Basics of Physiology and Interpretation, Gildea, et al., eds, Cleveland Clinic Publishers 2002, American Thoracic Society, 1995, Standardization of Spirometry, 1994 Update, Am. J. Respir. Crit. Care Med. 152:1107-1136, Pulmonary Function Testing, Guidelines and Controversies: Equipment, Methods, and Normal Values, Clausen and Zarins eds. New York: Academic Press, 1982, Becklake, et al., Am. Rev. Respir. Dis. 144:1202-1218, Crapo, et al., 1981, Am. Rev. Respir. Dis. 123:659-664 and Knudson, et al., 1983, Am. Rev. Respir. Dis. 127:725-34 each of which is incorporated herein by reference in its entirety.)

Sputa sampling and culture can also be used in conjunction with the methods of the invention to assist in diagnosis and to guide treatment. Sputum samples can be collected through expectorated sputa or sputum induction techniques. Additionally, exhaled breath condensate techniques can be useful for noninvasive collection of nongaseous components of the expiratory air, such as inflammatory mediators (EcoScreen; Jaeger, Würzburg, Germany). Microbial isolates from collected sputum can be identified by standard methods which are well known in the art. (See, Bogaert, et al., 2004, Infect. Immun. 72(2):818-23, Csoma, et al., 2002, Am. J. Respir. Crit. Care Med. 166(10): 1345-1349, Henig, et al., 2001, Thorax 56(4): 306-11, Gibson, 1998, Can. Respir. J. 5 Suppl A:22A-6A, each of which is incorporated herein by reference in its entirety.)

Pulmonary Infections

In certain embodiments, the invention provides methods of treating pulmonary infections. The pulmonary infection can be from any pathogen or combination of pathogens that causes pulmonary infections. In certain embodiments, the pathogen can be bacterial, viral, fungal or combinations thereof.

Bacterial pathogens treated by the methods of the invention can be community acquired, opportunist or a bioterrorist agent. Bacteria categorized as gram positive, gram negative, rods, cocci and atypical bacterial are encompassed by the scope of the present invention. Such bacterial pathogens include, but are not limited to, Staphlococci species, including S. aureus, S. epidermidis and S. saprophyticus; Streptococci species, including S. pneumoniae, S. sanguis, S. oralis, S. salivarius, S. mutans, S. pyogenes; Burkholeria cepacia, Chlamydia species including C. pneumoniae, Acinetobacter species, including Actinobacillus actinomycetemcomitans; Cardiobacterium hominis, Listeria monocytogenes, Branhamella catarrhalis (also sometimes classified as Moraxella catarrhalis), Klebsiella pneumoniae, Pseudomonas species, including P. aeruginosa; Escherichia coli, Enterobacter species, Proteus species, Serratia marcescens, Haemophilus species including H. influenzae and H. parainfluenzae; Legionella species including, L. pneumophila, L. micdadei, L. bozemanii and L. dumoffii; Mycobacterium species including M. pneumoniae, M. tuberculosis and M. bovis; and Gram negative bacilli, including Alkaligenes, Cardiobacterium and Eikenella species. Also included are atypical bacteria including, for example, Mycoplasma species, including M. pneumoniae. (See also, Merck Manual, 17th edition, Beers and Berkow, eds., 2004, Merck and Company, Critical Care Infectious Disease Textbook, Groenewegen and Wouters, 2003, Respir. Med. 97(7): 770-7, Rello, et al., eds., 2001, Kluwer Academic Publishers, Boston, Krugman's Infectious Disease of Children, 10th ed. Katz et al., eds. Mosby, St. Louis, incorporated herein by reference in their entireties).

Viral infections encompassed by the methods of the present invention include, but are not limited to, rhinovirus, respiratory syncytial virus, parainfluenza virus, influenza A and B viruses, adenovirus, picornavirus, varicella-zoster virus, Epstein-Barr virus, coxsackievirus, coronavirus, including SARS-associated coronavirus (SARS-CoV), Sin Nombre virus (causative agent for hantavirus) and cytomegalovirus. (See, Fete and Noyes 1996, Pediatr. Ann. 25:577-84; Feldman and Stokes, 1987, Semin. Respir. Infect. 2:84-94; Frank and Friedman 1988 Ann. Intern. Med. 109:769-71; Graman and Hall, 1989, Semin. Respir. Infect. 4:253-60; Greenberg 1991, Infect. Dis. Clin. North Am. 5:603-21; Jacobson and Mills, 1988, Clin. Chest Med. 9:443-8; Latham-Sadler and Morell 1996, Prim. Care. 23:837-48; Ljungman 1995, Semin. Respir. Infect. 10:209-15; website for Centers for Disease Control, National Center for Infectious Disease; Sethi, 2002, J. Respir. Dis. 23(4): 217-25, Tan, et al., 2003, Am. J. Med. 115(4):272-7, Hogg, 2001, Am. J. Respir. Crit. Care Med. 164 (10 Pt 2): S71-5 the contents of each of which are incorporated herein by reference in their entireties.)

In certain embodiments, the pulmonary infection treated by the methods of the invention are caused by fungi. Fungi encompassed by the methods of the invention include, Pneumocystis carinii, (now considered a fungus rather than a protozoan), Blastomyces species including B. dermatitidis; Cryptococcus species, Candida species including C. albicans; Aspergillus species, Histoplasma species including H. capsulatum; Coccidioides immitis, Sporothrix schenckii and Mucor species including, M. amphibiorum, M. circinelloides, M. hiemalis, M. indicus, M. racemosus, and M. ramosissimus. (See, Arnow et al., 1991, J. Infect. Dis. 164: 998-1002; Lentino et al., 1982, Amer. J. Epidemiol. 116:430-437; Dixon et al., 2004, Pharmacoeconomics 22(7): 421-33; Chiller et al., 2003, Infect. Dis. Clin. North Am. 17(1):41-57, viii; Introductory Mycology 4th ed. Alexopoulos, et al., eds. 1996, John Wiley, New York; Anaissie, et al., 1986, Cancer, 57: 2141-2145; Deresinski 2003, Semin. Respir. Infect. 18(3):216-9 each of which is incorporated herein by reference in their entireties.)

The methods of the invention can be used to treat pulmonary infections acquired through acts of bioterrorism. Agents identified as potential bioterrorist agents have been known prior to the threat of bioterrorism, however, their prevalence has been relatively low albeit their virulence high. Such infectious agents include Bacillus anthracis, smallpox variola, monkeypox viruses, Brucellosis species, Francisella tularensis (causative agent for tularemia), Yersinia pestis (causative agent for bubonic plague) and Ebola virus. See, Jones, et al., 2003, Clin. Microbiol. Infect. 9(9):984-6, Schriewer 2004, Methods Mol. Biol. 269:289-308, Guihot, 2004, Presse Med. 33(2): 119-22, Han and Zunt, 2003, Curr. Neurol. Neurosci. Rep. 3(6):476-82, Krug 2003, Antiviral Res. 2003 57(1-2):147-50, Whitley, 2003, Antiviral Res. 57(1-2):7-12 and Cunha, 2002, Clin. Microbiol. Infect. (8):489-503, each of which is incorporated herein by reference in their entireties.

The methods of the present invention provide for the treatment of pulmonary infection wherein the treatment reduces pathogen load in the lung. Certain lung diseases and allergies predispose subjects to acquiring a lung infection which can exacerbate their condition. Subjects with these lung diseases and allergies can be benefited by the methods of the present invention. For example, subjects with chronic obstructive pulmonary disease (COPD) or chronic bronchitis can experience acute exacerbations of their disease when an infection sets in. Frequent acute exacerbations in these diseases have a significant negative impact on the quality of life and pulmonary function of the subject. For example, subjects with COPD typically experience one to three acute exacerbations per year, accounting for roughly 500,000 hospitalizations each year in the United States alone. Acute exacerbations can occur with bacterial, fungal or viral pathogens, or combinations thereof. Bacterial pathogens which typically contribute to such acute exacerbations in subjects with COPD or chronic bronchitis include Haemophilus influenzae, Streptococcus pneumoniae and Pseudomonas aeruginosa, whereas viral pathogens typically include rhinovirus, respiratory syncytial virus (RSV) and influenza virus. See, Aaron, et al., 2000, Am. J. Respir. Cit. Care Med. 163:349-355, Bandi, et al., 2003, FEMS Immunol. Med. Microbiol. 37(1): 69-75, Bogaert et al., 2004, Infect. Immun. 72(2): 818-23, Khan, et al., 2003, J. Pak. Med. Assoc. 53(8): 338-45, Groenewegen and Wouters, 2003, Respir. Med. 97(7), 770-7, Soto and Varkey, 2003, Curr. Opin. Pul. Med. 2003, 9(2): 117-24, Miravitlles, 2002, Eur. Respir. J. Suppl. 36: 9s-19s, Lieberman, et al., 2002, Eur. Respir. J. 19(3): 392-7, each of which is incorporated by reference in its entirety.

Acute exacerbations also occur in subjects with asthma, caused most frequently by respiratory viral (rhinovirus, adenovirus, picornavirus and influenza virus) or bacterial (Chlamydia pneumonia, Mycoplasma pneumonia and Legionella species) infections. Acute exacerbation events in subjects with asthma typically involve both neutrophil and eosinophil infiltrations into the lung, however the neutrophil response is typically dominant. See, Wark, et al., 2001, Monaldi Arch. Chest. Dis. 56(5): 429-35, Jarjour, et al., 2000, J. Allergy Clin. Immunol. 105:1169-77, Wark, et al., 2002, Eur. Respir. J. 19(1):68-75, Wark, et al., 2002, Eur. Respir. J. 20(4): 834-40, Gibson, et al., 1999, Pediatr. Pulmonol. 28(4): 261-70, Tan, et al., 2003, Am. J. Med. 115(4):272-7, Twaddell, et al., 1996, Eur. Respir. J. 9(10):2104-8, each of which is incorporated by reference in its entirety.

Cystic fibrosis lung disease is characterized by an excessive inflammatory response, often associated with chronic Pseudomonas aeruginosa infection. Bronchoalveolar lavage in healthy subjects and subjects with cystic fibrosis lung disease showed that subjects with cystic fibrosis have elevated proinflammatory cytokines but negligible amounts of anti-inflammatory cytokine interleukin-10. Chmiel et al., 1999, Am. J. Respir. Crit. Care Med. 160:2040-2047. See also, Silva-Mejias, et al., 1995, Chest 108:942-945.

In certain embodiments, the methods of the invention provide for treatment of a subject having COPD, chronic bronchitis, pneumonia, pneumonitis, acute respiratory distress syndrome (ARDS), severe acute respiratory syndrome (SARS), sarcoidosis, cystic fibrosis, emphysema, asthma, smoker's cough, allergy allergic rhinitis, sinusitis or pulmonary fibrosis.

In certain embodiments of the invention, the methods provide for a reduction in the acute exacerbations of pulmonary symptoms in subjects with COPD, chronic bronchitis, pneumonia, pneumonitis, acute respiratory distress syndrome (ARDS), severe acute respiratory syndrome (SARS), sarcoidosis, cystic fibrosis, emphysema, asthma, smoker's cough, allergy, allergic rhinitis, sinusitis or pulmonary fibrosis.

Indications of pulmonary infection can include increased interleukin-1, interleukin-8, interleukin-10, protein and myeloperoxidase (MPO) cells in bronchoalveolar lavage. In certain embodiments, the methods of the invention provide for treating pulmonary infection wherein treatment can be evaluated via BAL. In certain embodiments, the methods provide for a decrease in interleukin-1, interleukin-8, protein and MPO cells in bronchoalveolar lavage from an afflicted subject. In certain embodiments, the methods provide for a decrease in pulmonary disease acute exacerbations as manifested by a decrease in coughing, a clearing of the sputum color, decrease in sputum quantity, decrease or freedom from dyspnea, improved oxygenation as indicated by skin or nailbed color or by pulse oximetry.

Anti-Selection Agents

Many cell adhesion molecules are known to be involved in the process of inflammation. At the site of inflammation, leukocytes first adhere to the vascular endothelial cells prior to the extravasation process. It is postulated that the members of a glycoprotein cell adhesion family, selectins, play crucial roles in the initial adhesion of leukocytes to endothelial cells, while other adhesion molecules such as integrins and members of the Ig superfamily are involved in a later process. Cell adhesion mediated by selectins and their carbohydrate ligands give rise to the tethering and rolling of leukocytes on endothelial linings, and this leads to the secondary firm adhesion and signal transduction mediated by integrins activated through the action of inflammatory chemokines such as interleukin-8 (IL-8) or MIP-1beta presented at the surface of endothelial cells. (Lasky, 1992, Science 258(5084):964-9).

The selectins have been implicated in the inflammatory process of disease states such as chronic obstructive pulmonary disease (COPD) (Noguera, et al., 2004, Chest 125(5):1837-42) asthma, (Sjosward, et al., 2004, Respiration 71(3):241-5) reactive arthritis, rheumatoid arthritis (RA), and sepsis (Kuuliala, et al. 2004, Scand. J. Rheumatol. 33(1):13-8) as well as to inflammatory processes in the skin, lungs and gut (See also, Ley and Kansas, 2004, Nat. Rev. Immunol. 4(5):325-36, Kuuliala, et al., 2004, J. Leukoc. Biol. 2004 May 3 [Epub ahead of print]).

The selectin family consists of three members, E-, P- and L-selectin. There are several significant differences between the characteristics of E- and P-selectin ligands. For instance, the P-selectin ligand on leukocytes is protease-sensitive, while that for E-selectin is highly resistant to protease treatments. It is still controversial whether L-selectin plays as equally important a role as E- and P-selectins in the recruitment of leukocytes in inflammation. L-selectin is suggested to be involved primarily in the physiological homing of lymphocytes to peripheral lymph nodes. P-selectin can be used as a sensitive marker in mild asthma. (See, Noguera et al., 2004, Respiration 71(3):241-5, Sjosward, et al., 2004, Scand. J. Rheumatol. 33(1):13-8, U.S. Pat. No. 6,204,007).

The methods of the invention provide for treatment of pulmonary infections by the administration of an anti-selectin agent. In certain embodiments of the invention, the anti-selectin agent inhibits leukocyte recruitment or migration, including the recruitment and migration of neutrophils, monocytes and eosinophils. In certain embodiments the anti-selectin agent can be an agent that competitively binds to a selectin so as to interrupt the interaction of a selectin with its ligand. In certain embodiments the anti-selectin agent can be an antibody, an antibody fragment, a peptide, a small molecule, a gene delivery of a selectin antagonist or combinations thereof.

Inhibition of leukocyte recruitment or migration can be determined in vitro by assay. In one embodiment, the assay can be ProteoFlow® assay as described in detail hereinbelow. In certain embodiments, the methods provide for a reduction in the average percent inhibition of rolling. In certain embodiments, the methods provide for about 40% inhibition of rolling, about 50% inhibition of rolling, about 60% inhibition of rolling or about 70% inhibition of rolling.

In certain embodiments the Biacore® assay can be used in the methods of the invention. The Biacore® assays provide for the detection and monitoring of biomolecular binding using surface plasmon resonance technology. These assays provide real-time quantitative data on binding interactions between biomolecules. Biacor AB, Piscataway, N.J.

In certain embodiments, the anti-selectin agent is an anti-selectin antibody or fragment thereof. An anti-selectin antibody can be monoclonal, polyclonal, or combinations thereof. An anti-selectin antibody can be humanized, chimeric or combinations thereof. Methods of making or producing antibodies and their fragments are known in the art and can be used to make the anti-selectin antibodies described herein.

In a preferred embodiment, an anti-selectin antibody is EL246, a monoclonal antibody secreted by a hybridoma, is selected as the anti-selectin agent. A sample of the hybridoma that secretes EL246 has been deposited in accordance with the Budapest Treaty under ATCC Accession No. HB 11049 and described in U.S. Pat. No. 5,756,095, which is herein incorporated by reference in its entirety. Other anti-selectin antibodies useful in the methods of the invention include E-selectin antibodies described in U.S. Pat. Nos. 6,204,007 and 5,632,991 and L-selectin antibodies described in U.S. Pat. No. 6,210,671. Other anti-selectin agents useful in the methods of the invention include peptides and peptide analogs as described in U.S. Pat. No. 6,111,065, carbohydrates and carbohydrate analogs as described in U.S. Pat. Nos. 5,962,660 and 5,830,871 or nucleic acid ligands as described in U.S. Pat. No. 6,280,932. The teachings of each of the foregoing patents are incorporated herein by reference in its entirety.

In some instances, it is desirable to prepare monoclonal antibodies from various mammalian hosts, such as mice, rodents, primates, humans, etc. A description of techniques for preparing such monoclonal antibodies can be found in, e.g., Stites, et al. (eds) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed) Academic Press, New York; and particularly in Kohler and Milstein (1975) in Nature 256: 495-497, which discusses one method of generating monoclonal antibodies. Each of these references is incorporated herein by reference. Summarized briefly, this method involves injecting an animal with an immunogen. The animal is then sacrificed, its spleen cells are then isolated and fused with myeloma cells. The result is a hybrid cell or “hybridoma” that is capable of reproducing in vitro. The population of hybridomas is then screened to isolate individual clones, each of which secretes a single antibody species reactive with the immunogen. In this manner, the individual antibody species obtained are the products of immortalized and cloned single B cells from the immune animal generated in response to a specific site recognized on the immunogenic substance.

Other suitable techniques involve in vitro exposure of lymphocytes to the antigenic species or alternatively to selection of libraries of antibodies in phage or similar vectors. See, Huse, et al. 1989, “Generation of a Large Combinatorial Library of the Immunoglobulin Repertoire in Phage Lambda,” Science 246:1275-1281; and Ward, et al. 1989, Nature 341:544-546, each of which is hereby incorporated herein by reference. The anti-selectin antibodies of the present invention can be used with or without modification, including chimeric or humanized antibodies. Patents teaching the making of various monoclonal antibodies include, U.S. Pat. Nos. 4,828,991, 5,665,357, 4,741,998, 5,863,796 and 6,056,957. These patents are incorporated herein by reference.

An anti-selectin antibody can be a composition comprising whole antibodies (e.g. both Fab and Fc) or composition comprising the antigen binding fragments of the antibodies. The antibody or portions thereof bind an antigenic determinant on one or more selectins, including for example, E-selectin and/or L-selectin. Alternatively, the anti-selectin antibody can bind to the same epitope on the selectin (e.g. E-selectin and/or L-selectin) as the monoclonal antibody EL246.

Methods of Administration and Pharmaceutical Compositions

The methods of the present invention provide for the treatment of pulmonary infections by the administration of pharmaceutical compositions comprising the anti-selectin agent. The methods of the invention can be practiced by any route of administration that provides a therapeutic amount of anti-selectin agent to the site of infection (e.g. lungs and lower respiratory tract). The dose of the anti-selectin agent is adequate to provide therapeutic amounts of anti-selectin compounds to the site of infection (e.g. lungs and lower respiratory tract).

The methods of the invention provide for administration of an anti-selectin agent. The administration can be in a single daily dose or divided daily doses, depending on a number of parameters including, the pharmacokinetic parameters of the anti-selectin agent, the presence of comorbid conditions in the subject, the route of administration and the severity of the illness. (See, Pharmacotherapy: A Pathophysiological Approach, 5th ed. DiPiro, et al., eds. 2002, McGraw Hill and Applied Therapeutics Handbook Koda-Kimble, et al., eds. 7th ed., Lippincott, Williams and Wilkins, incorporated herein by reference in their entireties). Regimens which include periodic administration of the agent every few days or weeks are also encompassed by the scope of the present invention.

A medical practitioner can discern the proper dose guided by clinical skill and the description provided herein. A typical daily dose can be from about 0.001 mg/kg body weight per day, to about 100 gm/kg body weight per day. In certain embodiments, a daily dose can be from about 0.01 mg/kg body weight per day to about 10 gm/kg body weight per day. In certain embodiments, a daily dose can be from about 0.1 mg/kg body weight per day to about 1 gm/kg body weight per day. During an acute exacerbation, for example in a subject with COPD, the increased work of breathing, ineffective cough, mucostasis, progressive hypoxemia or hypercarbia, confusion, and fatigue can result in overmedication or undermedication which can be prevented by dosing adjustments made by a medical practitioner.

The described methods provide for the administration of the anti-selectin agent for a duration sufficient to treat pulmonary infection. In certain embodiments, the treatment duration can be a one time dose or scheduled dosing over a period of time. In certain embodiments, the treatment duration can be days, weeks or even months. In certain embodiments, the treatment can be administered routinely (e.g. daily, every three days, every week, every two weeks or the like) or periodically (e.g. during acute exacerbations or periods of increased threat, for example, flu season). In certain embodiments ‘pulsed’ or ‘bolus’ doses of the anti-selectin agent can be administered to a subject in need, especially, for example, during an acute exacerbation. The amount and duration of such pulsed or bolus doses can be determined by a medical practioner.

The methods of the invention provide that the anti-selectin agent can be administered with other drugs, therapies and treatments. In certain embodiments, the anti-selectin agent can be administered with anti-infective drugs such as cephalosporins, penicillins, fluoroquinolones, erythromycins, tetracyclines, anti-viral drugs (such as amantidine, ribavirin and the like) and anti-fungal drugs (such as azole antifungals and amphotericin). In certain embodiments, the anti-selectin agent can be administered with drugs and therapies to treat or ameliorate accompanying symptoms of respiratory distress such as beta-agonists (albuterol and the like), steroids, anxiolytics, and pain relievers. In addition, respiratory therapy can be used in association with the administration of the anti-selectin agents to provide treatment for the pulmonary infection. (See, Drug Facts and Comparisons, updated monthly, Wolters Kluwer Company, St. Louis, Physicians Desk Reference, 58th ed., Medical Economics Company, 2003 each of which is incorporated herein by reference in its entirety.)

The route of administration is typically dictated by the pharmaceutical formulation of the active compound. The present invention provides that the pharmaceutical formulation can comprise the anti-selectin agent as the active ingredient in a pharmaceutically acceptable carrier suitable for administration and delivery in vivo. Because the antibody proteins can contain acidic and/or basic termini and/or side chains, the proteins can be included in the formulations in either the form of free acids or bases, or in the form of pharmaceutically acceptable salts. (See, Remington's Pharmaceutical Sciences, 19th ed. Remington and Gennaro eds., 1990, incorporated by reference herein in its entirety).

Injectable preparations can include sterile suspensions, solutions or emulsions of the anti-selectin agent in aqueous or oily vehicles. The compositions can also comprise formulating agents, such as suspending, stabilizing and/or dispersing agents. The formulations for injection can be presented in unit dosage form, e.g., in ampules or in multidose containers, and can contain added preservatives.

Alternatively, the injectable formulation can be provided in powder form for reconstitution with a suitable vehicle, including but not limited to sterile pyrogen free water, buffer, dextrose solution, etc., before use. To this end, the anti-selectin agents can be lyophilized. The stored preparations can be supplied in unit dosage forms and reconstituted prior to use in vivo.

For prolonged delivery, the active ingredient can be formulated as a depot preparation, for administration by implantation e.g., subcutaneous, intradermal, or intramuscular injection. Thus, for example, the active ingredient can be formulated with suitable polymeric or hydrophobic materials (e.g., as an emulsion in an acceptable oil) or ion exchange resins, or as sparingly soluble derivatives e.g., as a sparingly soluble salt form of the anti-selectin agent.

In one embodiment, the anti-selectin agent can be administered to the lungs via nasal or oral inhalation. For administration by inhalation, the active ingredient can be conveniently delivered in the form of an aerosol spray for delivery by pressurized packs or inhalers, for example, metered dose inhalers that can be conveniently used for the periodic treatment of chronic disease, or a nebulizer that can be used for the treatment of subjects suffering from an acute exacerbation. Such inhalers or pressured packs can use a Suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount (i.e. metered dose inhaler). Capsules and cartridges of a delivery vehicle, e.g. gelatin, for use in an inhaler or insufflator can be formulated containing a powder mix of the compound and a suitable powder base such as lactose or starch. (See also, Courrier et al., 2002 Crit. Rev. Ther. Drug Carrier Syst. 19(4-5): 425-98 incorporated herein by reference in its entirety.)

Alternatively, transdermal delivery systems manufactured as an adhesive disc or patch which slowly releases the anti-selectin agent for percutaneous absorption can be used. To this end, permeation enhancers can be used to facilitate transdermal penetration of the active ingredient.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For buccal administration, the compositions can take the form of tablets or lozenges formulated in conventional manner. For rectal and vaginal routes of administration, the active ingredient can be formulated as solutions (for retention enemas) suppositories or ointments.

The compositions can, if desired, be presented in a pack or dispenser device which can contain one or more unit dosage forms containing the active ingredient. The pack can for example comprise metal or plastic foil, such as a blister pack. The pack or dispenser device may be accompanied by instructions for administration.

In a preferred embodiment, the anti-selectin agent can be provided in a multi-use or single use vial of lyophilized powder available for reconstitution. A single use vial can preferably contain a dose appropriate and suitable for an average sized adult or pediatric subject.

In certain embodiments, the anti-selectin agent can be presented in a kit comprising one or more vials of the anti-selectin agent and syringes, or pre-filled syringes with typical doses (e.g. unit dose or unit of use) of the anti-selectin agents. The kits can comprise other compositions of the anti-selectin agents, for example, one or more metered dose inhalers or packages of solution or powder for use with a nebulizer or insufflator. The kits can further provide instructional videos, DVDs or written instructions for use and sharps container for appropriate disposal of syringes.

### EXAMPLES

### Example 1

### Effect of EL246 on Clearance of Pseudomonas aeruginosa in Sheep Lung

Introduction

EL246 mAb effectively blocks pulmonary inflammation induced by endotoxin, suggesting that it may be an effective drug for the treatment of pulmonary inflammatory disease. A concern with the use of anti-inflammatory drugs is that they may predispose the patient to secondary infections. EL246 was tested to determine whether it alters the ability of sheep to clear a pulmonary infection with Pseudomonas aeruginosa. The experimental approach was to establish pulmonary infection in sheep, treat i.v. with the test or control antibody, and monitor both leukocyte infiltrate levels and Pseudomonas counts in lung lavage samples taken over a one week period.

Methods

Organism and Culture Conditions. Pseudomonas aeruginosa strain PAO1 (a CF clinical isolate obtained from the Center for Biofilm Engineering, Montana State University) stock cultures were maintained at −80° C. For inoculum preparation, one to two colonies from a fresh R2A agar (Difco #1826-17) streak plate were transferred into 50 ml sterile Trypticase Soy Broth and incubated overnight with aeration (16-18 h, 180 rpm, 37° C.). The culture was centrifuged (10 min at 5,000×g, 4° C.), washed once in sterile Dulbecco's phosphate buffered saline (DPBS), and suspended in st-DPBS. The number of colony forming units (CFUs) in the suspension was determined via spread plate method from serial 10-fold dilutions (0.500 ml transferred to 4.5 ml dilution blanks and vortexed 45-60 sec) plated in duplicate onto R2A agar plates. Colonies were counted after overnight incubation (37° C.), and the original Pseudomonas suspension, which had been refrigerated overnight, further diluted to achieve the desired inoculum concentration for administering to sheep on Day 0.

Bronchoscope Inoculation and Lavage Sampling.

Animals consisted of 1-year old sheep. Baseline lavage samples were taken with a bronchoscope from each animal to determine initial neutrophil cell counts. The presence of preexisting infection determined by plating the baseline lavage sample as described below. All animals that had low neutrophil counts and no preexisting infection were rested at least a week and then infected into each lower lobe of the lung with 5×10¹⁰ CFUs of the Pseudomonas isolate described above emulsified in 0.25% (final concentration) agarose. Lavage samples were taken at days 1, 3, and 6 days after infection. Blood samples were also taken.

Processing of Lung Lavage Samples for Leukocyte Analyses.

Total leukocyte counts were made for each sample and the percentage of neutrophils was determined by FACs using mAbs directed against sheep neutrophils.

Processing of Lung Lavage Samples for Microbial Counts.

Two lung lavage samples from each sheep (one Right Lung and one Left Lung, bronchoscope-assisted) were placed on ice for transport and processed within four hours of collection. Samples were serially diluted (0.500 ml transferred to 4.5 ml dilution blanks) and plated in duplicate onto R2A agar plates, and plates incubated overnight at 37° C. Resultant microbial colonies were assessed and categorized as P. aeruginosa by appearance (the PAO1 isolate appears greenish yellow on R2A) or non-Pseudomonas (confirmed by Gram staining). The bacterial cfu/ml lavage was determined from R2A agar plates containing 30-300 colonies whenever possible. Lavage samples for each sheep were processed: pre-experiment (e.g. Days −6 to 0), Day 1, Day 3, and Day 6.

Antibody Administration.

When the Day 1 bacterial counts became available on Day 2, sheep with established Pseudomonas infections were treated i.v. on Day 2 with 1 mg/kg mAb EL246, control irrelevant mAb (GD3.5), or nothing.

Results

Infection was followed by a rise in temperature within hours and general lethargy in all animals. Temperatures remained elevated through at least 3 days following infection. The fever response within the first few hours following inoculation was not predictive of active infection at 24 hours in all animals. As such, CFU counts in lavage samples at 24 hours was the only way to ensure established infection in animals that were subsequently treated with mAb.

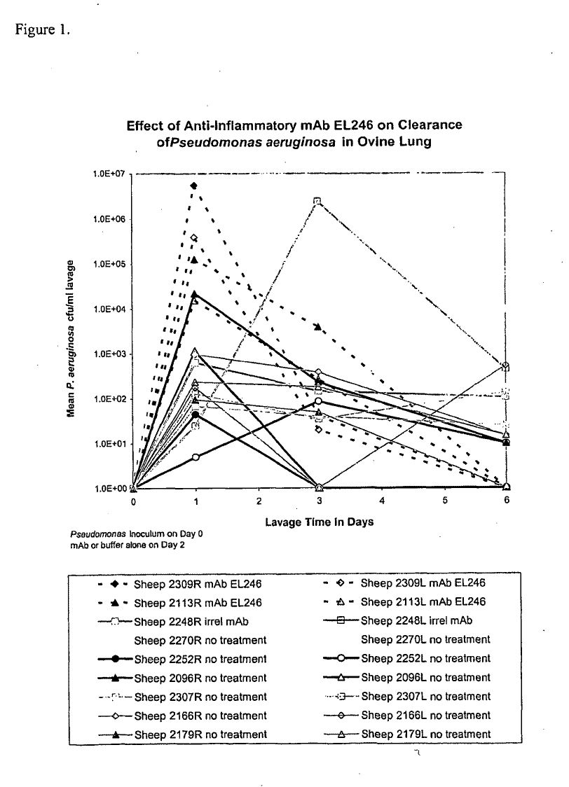

Leukocyte counts in the infected lavage samples were highly variable, due to variation in the sampling of each lobe (data not shown). As such, changes in the percentage of neutrophils were used as a comparison of inflammation in the different samples. Animals showed enhanced clearance of the bacteria following EL246 treatment (FIG. 1). Additionally, there was a slight decrease in the percentage of neutrophils in the EL246 treated group compared to the controls at day 6 following infection.

mAb EL246 treatment did not exacerbate and, in some cases, aided clearance of Pseudomonas aeruginosa from the lung. All EL246 treated animals cleared infection and showed no obvious clinical signs of infection by day 6.

Statistics

Effect of Anti-Inflamatory mAb EL246 on Clearance of Pseudomonas aeruginosa in Ovine Lung.

These data (FIG. 1) were analyzed using a mixed effects linear model, where day and treatment were considered fixed effects, and sheep and side within sheep were considered random effects. Pre-experiment lavage counts were not included in the analysis due to the fact that all values were zero. The slope of the lines was compared between treatment groups to look for statistical significance. Using this model, the line corresponding to anti-inflammatory mAb EL246 has a very strong negative slope which is statistically significantly different from both the slope of the line corresponding to no treatment (p=0.0001) and the slope of the line corresponding to the irrelevant mAb treatment (p=0.0007).

Summary

Here Applicants show that EL246 at a 1 mg/kg dose surprisingly enhances clearance of an established Pseudomonas aeruginosa infection in the sheep lung. EL246 treated animals cleared the infection with statistical significance, compared with untreated animals or isotype-matched negative control mAb treated animals.

### Example 2

### Effect of EL246 on Clearance of Non-Pseudomonas aeruginosa Bacteria in Sheep

The effect of EL246 treatment on clearance of bacterial organisms other than P. aeruginosa from the lung was noted in the sheep. The pulmonary infection model with Pseudomonas inoculation of sheep and bacterial count monitoring with the microbial spread plate method was as described in Example 1. The Pseudomonas aeruginosa PAO1 strain colonies appeared as distinct, translucent green-blue colony growth with spreading margins on R2A agar. The appearance of other bacterial colonies was observed for some sheep lavage samples, indicating that other genera of bacterial organisms were likely. These other colonies were counted and recorded separately in order to assess clearance of these non-Pseudomonas aeruginosa bacterial types in the presence of antibody treatment. In some cases, the only bacteria isolated from particular lavage samples were non-Pseudomonas. Described below are two example colony types and Gram stain reactions recorded for non-Pseudomonas organisms found in samples. A Gram stain of the Pseudomonas aeruginosa PAO1 isolate utilized in these studies demonstrated the classic Gram negative morphology expected for Pseudomonas: white, punctuate, small round colonies—long, slender Gram positive rods; white, asteroid, and mucoid colonies—very large, long Gram positive rods with very apparent exopolysaccharide halo that stains pink with the safranin counter-stain. Isolation of a strong exopolysaccharide-producer from lavage samples suggests the potential to form biofilms in vivo.

Results

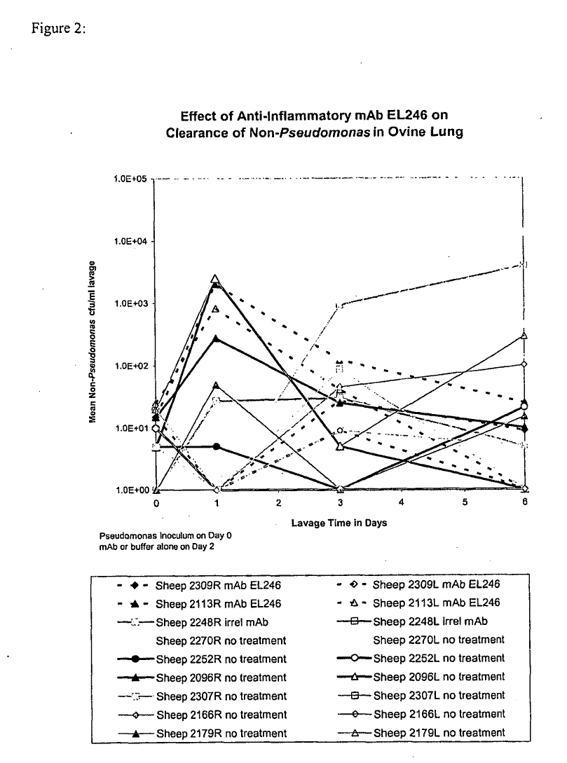

mAb EL246 treatment promoted clearance of non-Pseudomonas organisms from infected sheep, similar to the clearance of Pseudomonas. Sheep treated with control antibody or buffer only usually demonstrated greater non-Pseudomonas counts and higher counts later in the experimental course than sheep treated with mAb EL246. Results showing these non-Pseudomonas clearance effects are provided in FIG. 2. For example, five of seven control animals shown in FIG. 2 (irrelevant mAb or no treatment) demonstrated rising bacterial counts on day 6 compared with either days 1 or 3, whereas all EL246 treated animals showed fewer cfu/ml lavage on day 6 compared to days 1 or 3.

### Example 3

### Non-Human Primate Acute Lung Exacerbation Model

Summary

An LPS aerosol challenge response in the lung was used to evaluate the effectiveness of EL246 treatment on acute exacerbation associated inflammatory cell recruitment to the lung of Cynomolgus monkeys.

Background

It is known that during acute exacerbations of COPD and asthma that both neutrophils and macrophages are recruited to the lung. This model, using Escherichia coli lipopolysaccharide (LPS), provided for evaluation of the magnitude and relative numbers of inflammatory cells in the bronchoalveolar lavage (BAL) as primary endpoints. For this, total numbers of inflammatory cells, neutrophils and macrophages were determined at the 12 hour time point. In addition, other secondary parameters were measured including IL-8, myeloperoxidase, serum proteins and IL-1beta. IL-8 provides a measurement of chemokine promoting inflammatory cell recruitment; myeloperoxidase, the extent of neutrophil driven tissue damage potential; serum protein, the actual vascular wall damage resulting in serum leakage into the lung; and IL-1beta, an assessment of the overall pro-inflammatory state. Overall, these measurements provide a relative severity index of inflammation and are indicative of the pro-inflammatory state of the lung.

Experimental Design and Methods

Monkeys were treated with 1 mg/kg mAb EL246 at (−) 15 minutes relative to LPS challenge and again at 1 hour post LPS challenge. E. coli LPS was delivered at a concentration of 0.4 mg/kg at time “0” via aerosol. Monkeys acted as their own internal controls, being challenged a month earlier with LPS with no mAb treatment. BAL was collected at 12 hours post LPS challenge for measurement of primary and secondary parameters. Inflammatory cells were counted by flow cytometry and secondary indicators were quantified in ELISA and biochemical assays.

Results

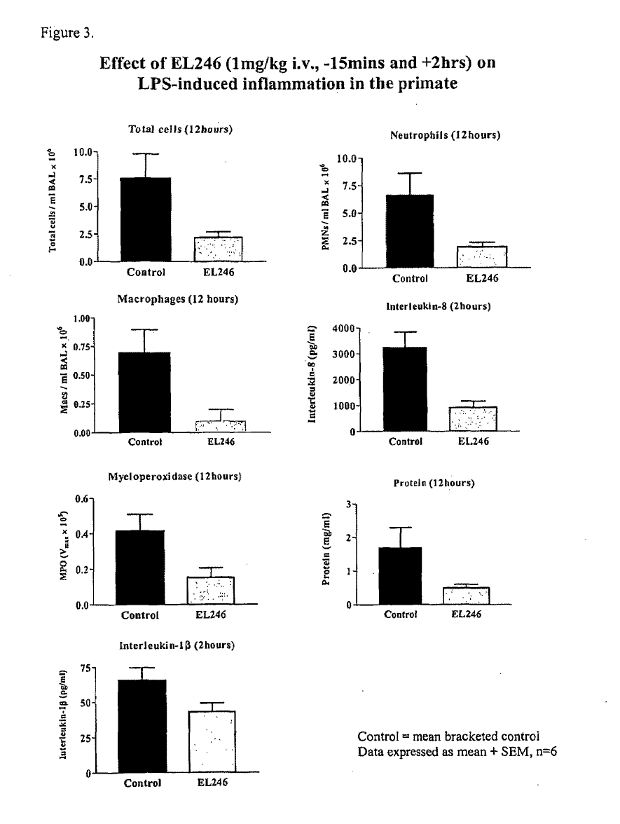

Twelve hour results for BAL determinations are shown in FIG. 3. The primary end point measurements showed a strong reduction of inflammatory cell recruitment relative to control. Total cells were reduced by more than two thirds, neutrophils by 70% and macrophages by greater than 80%. This reduction points to a major decrease in potential for cell mediated oxidative damage to the lung which is borne out in a secondary parameter where myeloperoxidase has been shown to also be reduced by roughly 70%. Other secondary indictors, IL-8 and serum protein, were also reduced by 60-70%. Surprisingly, IL-1 beta was also reduced by 25% showing that reduction of inflammatory cell recruitment might also aid in reducing the amplification of the pro-inflammatory state.

Summary

EL246 is very effective in reducing recruitment of neutrophils and macrophages, as is commonly seen in acute exacerbations of diseases like COPD and asthma. This suggests that an E- and L-selectin blocking strategy using EL246 may contribute greatly to reduce the severity of acute exacerbations.

### Example 4

### Real-Time In Vitro Shear Assay, ProteoFlow®, a Physiologically Relevant Assay System to Validate Adhesion Molecule and Chemokine Inhibitors

Applicants describe a progressive screening platform, ProteoFlow®, for the identification and validation of antagonists of leukocyte adhesion and chemokine signaling. Immobilized adhesion molecules on the internal surface of capillary tubes, endothelial cells or recombinant adhesion molecule expressing transfectants grown inside the capillary tubes are used to mimic a blood vessel. At physiologic shear rates, these substrates mediate tethering and rolling of human cells and cell lines. This assay is useful for testing the prophylactic (pre-adhesion treatment) and therapeutic (post-adhesion treatment) efficacy of potential new drug compounds that interfere with leukocyte trafficking. As examples of the utility of the ProteoFlow® system, Applicants demonstrate the ability to discern subtle differences in efficacy of a proprietary selectin inhibitor and the level of complexity that can be achieved by establishing chemokine-induced T cell adhesion to inflamed endothelial cells. In the first example, Applicants show that an anti-E+L-selectin antibody (EL246) was more effective (IC50<1 μg/ml) at reversing human myeloid U937 cell rolling on purified E-selectin chimera than when compared to a CHO cell line expressing E-selectin. When the ProteoFlow® system was fully human, i.e. human neutrophils rolling on HUVECs, the IC50 was higher than 2 μg/ml. In the second example of the utility of ProteoFlow®, immobilization of the chemokine, CXCL12 (SDF-1α), induced adhesion of Jurkat cells to TNFα activated HUVECs through α₄ integrins. As demonstrated in these examples, the ProteoFlow® system can provide visual and quantifiable evidence for ranking the effectiveness of drug candidates and allows for determination of dose assessment such as IC50.

Leukocyte extravasation is both a normal physiological process necessary for efficient immune surveillance and an essential component of inflammatory response to injury, infection and allergy (Butcher and Picker, 1996 Science 272:60-66, Von Andrian and MacKay, 2000 N. Engl. J. Med. 343:1020-1034). Adhesion proteins expressed by both the leukocyte in the blood and the endothelial cell control these interactions (Butcher, 1991 Cell 67:1033-1036, Bevilacqua, 1993, Ann. Rev. Immunol. 11:767-804.) In vivo measurements have shown that cells in the blood travel at surprisingly high velocities. Even in the reduced flow associated with the capillary and post-capillary beds, velocities of 500-5000 μm/sec are common (Ley, et al., 1988, Pflugers Arch. 412:93-100). Analyses of leukocyte/endothelial cell interactions demonstrate that specific classes of adhesion proteins preferentially mediate binding under shear versus static conditions (Butcher, 1991 Cell 67:1033-1036, Ley, et al., 1991, Blood 77:2553-2555, Lasky, 1992, Science 258:964-969, Abbassi, et al., 1993, J. Clin. Invest. 92:2719-2730, Von Andrian, et al., 1991, Proc. Nail. Acad. Sci. USA 88:7538-7542, Lawrence and Springer, 1991, Cell 65:859-873, Springer, 1990, Nature 346:425-434, Lawrence and Springer, 1993, J. Immunol. 151:6338-6346, Tozeren. and Ley, 1992, Biophys. J. 63:700-709). For example, many well characterized static adhesive interactions between leukocytes and endothelial cells, those mediated by the beta-2 integrins (LFA-1, MAC-1), were found not to take place, unless preceded by a selectin interaction, in in vitro assays under high shear conditions designed to reflect blood flow (Lawrence and Springer, 1991, Cell 65:859-873, Von Andrian, et al., 1992, Am. J. Physiol.: Heart Cir. Physiol. 263:H1034-H1044). In contrast, adhesion molecules have been identified which preferentially function under shear.

The best example of shear-dependent adhesion proteins is the family of leukocyte and endothelial cell molecules called selectins (Bevilacqua, 1993, Ann. Rev. Immunol. 11:767-804, Lasky, 1992, Science 258:964-969, Bevilacqua, et al. 1991, Cell 67:233). One leukocyte selectin (L-selectin) and 2 vascular selectins (E- and P-selectin) have been defined. In addition to endothelial cells, platelets also express P-selectin. L-selectin is constitutively expressed by leukocytes, whereas, stimulation of endothelial cells with immune cytokines, histamine or traumatic insult is required to induce E- and P-selectin. In addition to selecting, other adhesion proteins, such as VCAM-1 on cytokine-stimulated endothelial cells (Berlin, et al., 1995, Cell 80:413-422), MADCAM-1 on high endothelial venules of the gut, PNAd-1 on high endothelial venules in peripheral lymph nodes (Berlin, 1993 Cell 74:1-20), high carbohydrate-containing, mucin-like molecules (Levinovitz, et al. 1993 J. Cell. Biol. 121:449-459, Moore, et al., 1992, J. Cell Biol. 118:445-456), and some integrins (α₄β₁ and α₄β₇) Berg, et al., 1993, Nature 366:695-698, Alon, et al., 1995, J. Cell. Biol. 128:1243-1253) also mediate shear-dependent interactions. In most instances, rolling of the leukocyte along the endothelial monolayer represents shear-dependent adhesive interactions. These rolling interactions can cause >1000-fold reductions in the velocity of the leukocyte. It has been hypothesized that the rolling of leukocytes along the vascular endothelium allows an endothelial cell presented chemokine to bind its cognate receptor, thereby inducing other adhesive interactions to take place, which “cement” the binding of the leukocytes to the vascular endothelium (Springer, 1994, Cell 76:301-314). Migration through the vascular lining then takes place. Thus, 3 steps are involved in successful arrest of leukocytes along the vessel wall: 1) shear-dependent capture and rolling, 2) activation dependent adhesion strengthening (slow rolling), followed by tight adhesion, and 3) transendothelial cell migration. Inducible adhesion molecules on the endothelium are involved in each of these steps. All 3 steps are required for the effective recruitment of inflammatory leukocytes to sites of injury or infection, or the seeding of lymphoid tissues (Springer, 1994, Cell 76:301-314 and Bochner, 2000, J. Allergy Clin. Immunol. 106:817-828). The establishment of this model has come, in part, from laborious and costly animal experimentation.

Because of the difficulties and cost of animal experimentation, several laboratories have pursued the development of in vitro shear assays. The majority of research laboratories have used a planar chamber to grow endothelial cells and then connect the chamber to a syringe pump to deliver cells or other reagents (Jones, et al., 1994, J. Clin. Invest. 94:2443-2450, Luscinskas, et al., 1994, J. Cell Biol. 125:1417, Alon, et al., 1994, J. Cell Biol. 127:1485-1495). ProteoFlow® is an in vitro assay system that accurately simulates human vascular flow conditions as described below and in accord with protocols described in previous publications (Berlin, et al., 1995, Cell 80:413-422, Berg, et al., 1993, Nature 366:695-698, Bargatze, et al., 1994, J. Immunol. 152:5814-5825, Egger, et al. 2002, J. Pharmacol. Exp. Ther. 302:153-162, Glee, et al., 2001, Infect. Immun. 69:2815-2820, Bargatze, et al., 1994, J. Exp. Med. 180:1785-1792, Jutila, et al., 1994, J. Immunol. 153:3917-3928, incorporated herein by reference in their entireties). This simulated microenvironment is an excellent tool for evaluating the molecular, cellular, and vascular surface interactions associated with inflammatory and infectious diseases.

The ProteoFlow System

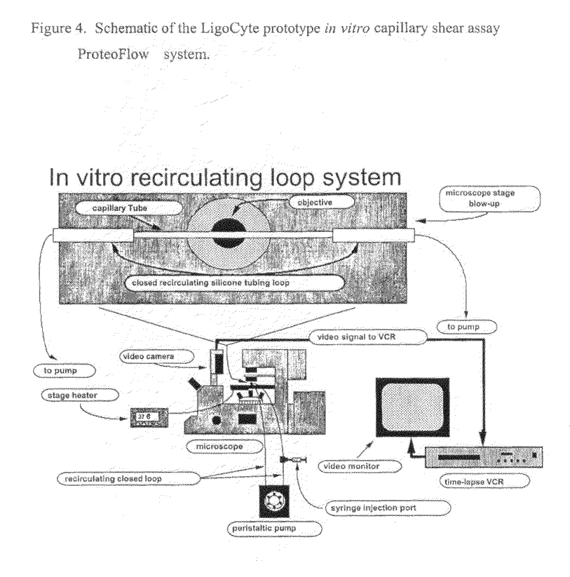

A central feature of the ProteoFlow® shear assay system is an artificial vessel that is created by growing endothelial cells, growing transfected cell lines expressing single or multiple adhesion molecules or adhering purified adhesion molecules on the internal surface of small diameter glass capillary tubes. The “vessels” are integrated into a loop system in which fluid can be recirculated via a peristaltic pump (FIG. 4). Cells are injected into the system, and their interaction with the endothelial cell monolayer is monitored by video-microscopy. Agents can be infused into the assay and their effect on leukocyte-endothelial cell interactions readily measured. The shear forces generated in the capillary tube are similar to the shear factors measured in blood vessels (Perry and Granger 1991, J. Clin. Invest. 87:1798-1804).

Assay Setup

Leukocyte/Endothelial Cell Interactions: The analyses of leukocyte/endothelial cell shear dependent interactions were conducted as described earlier (Berg, et al., 1993, Nature 366:695-698, Bargatze et al., 1994, J. Immunol. 152:5814-5825, Berg et al., 1991, J. Cell Biol. 114:343-349, each of which is incorporated herein by reference in their entireties). The human pro-monocytic cell line, U937 (ATCC), has been extensively used as a surrogate to study human leukocytes. U937 cells express the E-, P- and L-selectin ligand PSGL and roll on selectins (Norgard, et al., 1993, J. Biol. Chem. 268:12764-12774, Hirata, et al., 2000, J. Exp. Med. 192:1669-1676, Yang, et al., 1999, Thromb. Haemost. 81:1-7, Larsen, et al., 1992, J. Biol. Chem. 267:11104-11110). EL246 is a novel antibody that binds to a conserved epitope on both L- and E-selectin (Jutila, et al., 1992, J. Exp. Med. 175:1565-1573). Human umbilical-cord endothelial cells (HUVEC; Cambrex Corp.), which are Factor VIII and LDL-receptor positive [cultured in endothelial-cell growth media (Clonetics, EGM)] or P- or E-selectin cDNA transfected CHO cells were grown to confluency on the internal surface of sterile glass capillary tubes (Drummond Scientific, Broomall, Pa.) 24 hours prior to shear experiments. Four hours prior to the assay, the endothelial cells were treated with TNFα (1 μg/ml) to induce E-selectin, ICAM-1 and VCAM-1 expression. Tubing attached to the ends of the glass capillary tube form a closed loop in which media and cells are to be recirculated; the tube was then mounted on an inverted microscope. Using a variable speed peristaltic pump (see FIG. 4), flow was regulated to simulate in vivo blood flow shear conditions (1-3 dynes/cm²). The circulation loop allows multiple infusions, via an injection port, of various mAbs/other test compounds during the continuous recirculation of leukocytes across the interactive surface of the HUVECs.

An inverted microscope-video capture system (FIG. 4) was used to survey the entire length of the HUVEC monolayer and high resolution phase contrast recording of the interactive field was performed for subsequent analysis. Leukocyte cell lines were infused into the system at a 4×10⁶ cell/ml concentration in sterile Ca⁺⁺, Mg⁺⁺ containing HEPES buffered (20 mM) DMEM or RPMI (pH 7.0) plus 2% FBS or human plasma. Rolling interaction was established and continuously monitored for at least 8 min while being videotaped; during that time, control or experimental conditions were established and maintained. The leukocyte/endothelial-cell interactions were observed and videotaped for an additional 8 min. The number of cells rolling on the substrate (chimeric adhesion molecules) was monitored before and after the injection of adhesion modifiers and determined by individual frame analysis of the recording. In experiments with selectin-transfectants and activated HUVECs the control or test compounds were infused with the interacting cells and the interaction monitored for a total of 10 min. Data was recorded as the number of rolling cells within the field of view versus time.

Results and Discussion

E-Selectin Mediated Rolling of U937 Cells in the ProteoFlow System.

Effect of an anti-E-plus L-selectin mAb, EL246, on U937 rolling oil recombinant chimera: One of the earliest steps in leukocyte recruitment, both in physiological recirculation and migration into inflamed sites, is the capture of the fast flowing leukocyte and establishment of a slow rolling interaction (Butcher, 1991, Cell 67:1033-1036). This capture and rolling is mediated by the selectin family of adhesion molecules (Bevilacqua, 1993, Ann. Rev. Immunol. 11:767-804, Lasky, 1992, Science 258:964-969). Inhibition of this first step abrogates the leukocytes ability to firmly adhere to and transmigrate through the endothelial layer thus providing a therapeutic target (Von Andrian, et al., 1991, Proc. Natl. Acad. Sci. USA 88:7538-7542, Bargatze, et al., 1995, Immunity 3:99-108). The ProteoFlow® system allows one to model this initial interaction in vitro and evaluate modulators of this interaction. The human pro-monocytic cell line, U937, which expresses the E-selectin ligand, PSGL 34 (Norgard, et al., 1993, J. Biol. Chem. 268:12764-12774, Hirata, et al., 2000, J. Exp. Med. 192:1669-1676, Yang, et al., 1999, Thromb. Haemost. 81:1-7, Larsen, et al., 1992, J. Biol. Chem. 267:11104-11110), was evaluated for its ability to interact with recombinant human E-selectin under shear. Recombinant E-selectin-human IgG chimera (R&D systems) was immobilized on the internal surface of glass capillary tubes coated with an anti-human IgG. The capillary tube was incorporated into the ProteoFlow® system as described in FIG. 4.

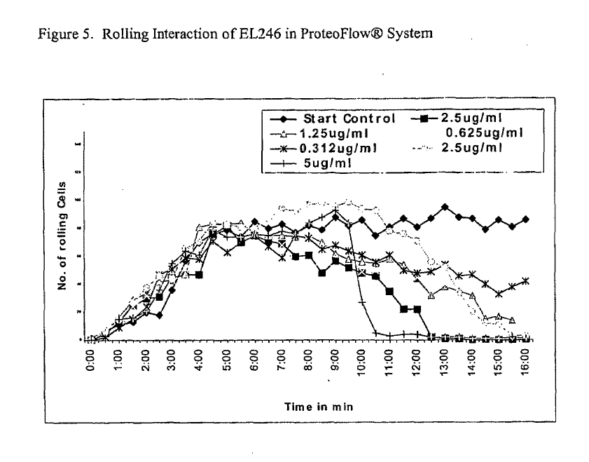

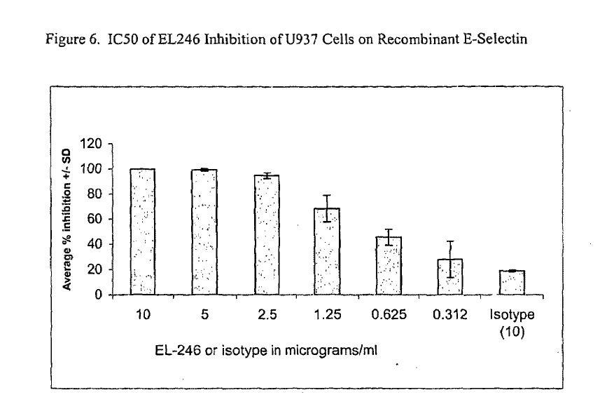

Flow was established to generate the physiological venular shear stress of 2 dynes/cm². U937 cells injected into the loop interacted with the immobilized E-selectin displaying a “rolling” behavior as is known to occur in vivo. The numbers of rolling cells increase from time of injection to ˜6 min and then plateaus (FIG. 5). EL246 mAb or isotype control mAb was injected 8 min after cell infusion and the interaction monitored for an additional 8 min. As shown in a representative experiment in FIG. 5 and summarized in FIG. 6, EL246 completely reversed the rolling interaction at 5 μg/ml. The reversal was dose dependent and unaffected by an isotype matched irrelevant mAb. EL246 reversed the rolling of U937 on recombinant E-selectin with an IC50 of ˜0.6 μg/ml (FIG. 6). Thus the ProteoFlow® system allows the visualization of selectin mediated leukocyte rolling and the evaluation of modulators of this interaction. In this in vitro setting, using a defined selectin chimera as a substrate, an inhibitor of L- and E-selectin appears to be quite potent.

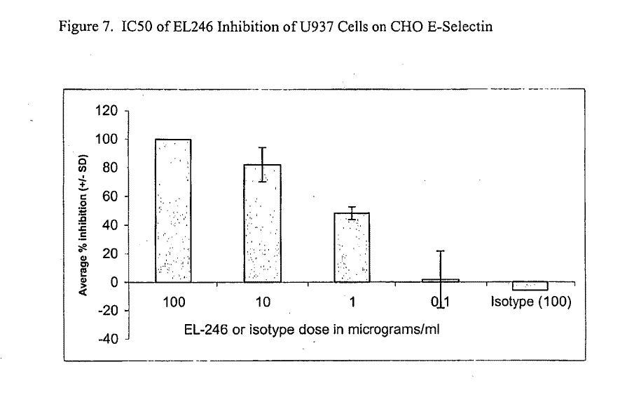

Effect of mAb EL246 on U937 cell rolling on CHO cells expressing human E-selectin: E-selectin is expressed on the intimal surface of activated endothelial cells (Lasky, 1992, Science 258:964-969). Therefore, to more realistically reflect the selectin-leukocyte interaction thought to occur in vivo, CHO cells transfected with human E-selectin cDNA were used as the substrate in the ProteoFlow® loop. CHO cells stably expressing human E-selectin were grown on the internal surface of sterile glass capillary tubes in the ProteoFlow® system. Interaction of U937 cells with E-selectin expressed on CHO cells was different from that which occurs on recombinant selectin. U937 cells rolled and then most of them stuck to the transfected CHO cells. The sticking was probably due to crossreactivity of human integrins on the U937 cells binding to counter-receptors on the CHO cells. Since anti-selectin antibodies will not reverse an integrin-mediated adhesion, the U937 cells were pre-treated with nab EL246 prior to injection into the ProteoFlow®R loop. As shown in FIG. 7, mAb EL246 inhibited the binding of U937 cells to CHO-E-selectin transfectants. However, the IC50 for inhibition was higher (>1 μg/ml) for inhibiting a cell based adhesion event as opposed to reversal of binding to purified adhesion molecule (˜0.6 μg/ml). These data suggest that efficacy of adhesion molecule inhibitors will vary depending on nature and type of presentation of the adhesion molecule chosen for evaluation when tested under shear.

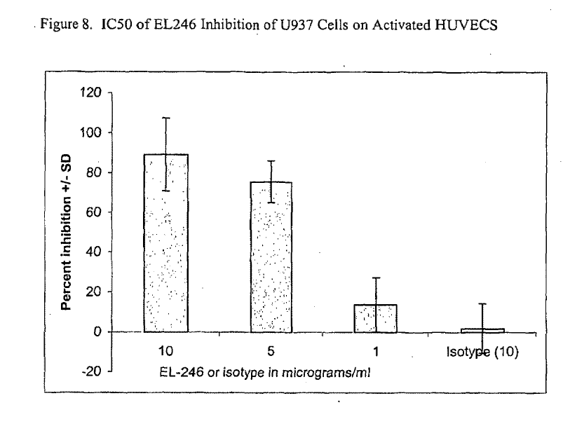

Effect of anti-E-selectin mAb on human neutrophils rolling on activated HUVECs: The critical adhesive interactions between leukocytes and endothelium involve both a selectin mediated rolling followed by integrin mediated sticking both of which are prerequisite to transmigration (Butcher, 1991, Cell 67:1033-1036, Von Andrian, et al., 1992, Am. J. Physiol. Heart Circ. Physiol. 263:H1034-H1044). The two in vitro shear assays described above do not have the complete complement of adhesion molecules required for leukocyte endothelial interactions that occur in vivo. Therefore, an assay was set up wherein HUVECs were grown on the internal surface of glass capillary tubes to mimic a “vessel” and activated the cells with TNFα (1 ng/ml) for 4 hours as a surrogate inflammatory stimulus. The four-hour activation induces the expression of E-selectin and the integrin ligands ICAM and VCAM-1 on HUVECs (data not shown). To completely simulate an in vivo leukocyte-endothelial interaction, peripheral blood neutrophils (PMNs) were isolated from healthy donors and injected into the ProteoFlow® loop containing the activated HUVECs. The efficacy of the anti-E-/L-selectin mAb EL246 in this in vitro system was then tested, which more realistically mimics conditions occurring in vivo. As described for the CHO transfectants (above), PMNs roll briefly and stick to the activated HUVECs. EL246 inhibited the PMN binding to HUVECs in a dose dependent manner (FIG. 8). An irrelevant isotype matched mAb had no effect on PMN-HUVEC interaction. However, the IC50 to inhibit PMN-HUVEC interaction was >2 μg/ml (FIG. 8). Thus, reproducing most of the adhesive components known to be required for leukocyte-endothelial interaction in vivo allows discrimination of subtle differences in drug efficacy. These data show that the same inhibitor has differing IC50 values (˜0.6 μg/ml to ˜2 μg/ml), and the efficacy is lower as the complexity of the ProteoFlow® system made more representative of the conditions thought to occur in vivo.

Conclusion

One of the most dynamic microenvironments in which critically important cell-cell interactions occur is the vascular system of animals. Current approaches in cell-cell adhesion analyses rely, in many instances, on antiquated in vitro assays that are not reflective of the in vivo microenvironment and/or time-consuming expensive animal testing which is quickly losing public favor. Antiquated in vitro assays primarily measure adhesion in static assays, but it is now known that interactions must be measured under shear forces that approximate those defined in vivo to gain a full appreciation of an interaction that takes place.

Until the recent use of in vitro shear approaches in academic labs, the primary means of determining the physiological relevance of a given adhesion event has been to perform extensive animal testing. The results of in vivo methods provide only an indirect endpoint measurement or when using direct in situ visualization, require even more animals to achieve statistically significant results. However, from these laborious in vivo studies, it has become quite clear that to gain a full appreciation of cell-cell interactions in blood, they must be examined under the shear forces associated with blood flow. There are many examples of adhesion systems defined under static conditions, which were later shown not to function when shear is applied. Here is described a progressive drug-screening tool, ProteoFlow®, which is an in vitro assay system that accurately simulates human vascular flow conditions. This simulated microenvironment is an excellent tool for evaluating the molecular, cellular, and vascular surface interactions associated with inflammatory and infectious diseases.

Through the use of live human cells in simulated vascular flow models the ProteoFlow system can provide visual and quantifiable evidence of the effectiveness of drug candidates. By examining drug candidates in this microenvironment, research scientists can quickly and precisely determine drug efficacy in a very cost effective manner. ProteoFlow® is a drug development tool that can quickly and cost effectively identify and optimize drug candidates for inflammatory diseases and infectious diseases.

### Example 5

### Determination of Estimated Human I.V. Dosing

Together, a human neutrophil activated-HUVEC recruitment ProteoFlow® analysis model coupled with a primate inhaled LPS lung inflammation systemic model were used to provide an estimate of dosing levels that would be effective in treating neutrophil driven lung inflammation with humanized EL246 mAb in humans.

Experiments run in ProteoFlow® established that an effective dose of EL246 could be reached in vitro for completely disrupting rolling recruitment of neutrophils under simulated physiologic blood flow conditions. Using this same method in a dose response protocol allowed establishment of an IC50 of 2.5 μg/ml for the EL246 mAb. In parallel, EL246 tested in the primate lung inflammation model at a dose of 1 mg/kg, was shown to inhibit an average of 50% of the neutrophil lung recruitment in response to the LPS inflammatory stimulus.

Comparison of the estimated dose from both of these studies allowed a ratio of effective ProteoFlow® determined dose versus effective in vivo primate dose to be established. As the ProteoFlow® system determinations are drug quantity/unit volume based, establishment of the ratio is highly dependent upon an estimate of blood volume/kg body weight in primates. Using this method of estimation, based upon accepted standards (primate weight (kg)×60=mLs total blood volume), the ratio calculated is 6.75. Thus, by establishing an IC50 in ProteoFlow® and the calculated multiplier of 6.75 the means are established for predicting an EC50 for humanized antibodies derived from EL246 to be used for treatment of human lung inflammation. Because standard weight to blood volume estimates for humans versus primates varies (human weight (kg)×71.43=mLs total blood volume), a human conversion factor to establish the human EC50 would be slightly lower at a ratio of 5.6 times the ProteoFlow® IC50 determination.

TABLE 1
Relationship of ProteoFlow ® EL246
IC50 Data to Whole Body Dosing

| | | | |
|---|---|---|---|
| | | | Average |
| | | | human dose, |
| Factor* | μg/ml | mg/L | 70 kg |

ProteoFlow ® Study

| | | |
|---|---|---|
| IC50 of human neutrophils | 2.5 | 2.5 |
| rolling on human umbilical | | |
| vein endothelial cells | | |

Primate Study

| | | | |
|---|---|---|---|
| Tested effective estimated EC50 | | 16.7 | 16.7 |
| dose (1 mg/kg) | | | |
| animal weight (kg) × 60 = mLs | 6.7 | | |
| total blood volume (60 mL/kg) | | | |

Whole Body, human

| | |
|---|---|
| Human weight (kg) × 71.43 = mLs | 5.6 |
| total blood volume (71.34 mL/kg) | |

Estimated Dosing

| | | | | |
|---|---|---|---|---|
| Average dosing based on primate | 6.7 | 16.7 | 16.7 | 85 mg/5 L |
| blood volume/kg | | | | (1.2 mg/kg) |
| Average dosing based on human | 5.6 | 14.0 | 14.0 | 70 mg/5 L |
| blood volume/kg | | | | (1 mg/kg) |

*Blood volume conversion factor: (ProteoFlow ® IC50) × (6.7 for primate or 5.6 for human)
**Average human weight = 70 kg; Average human blood volume = 5 Liters.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

We claim:

1. A method of treating or preventing a pulmonary infection comprising administering to a patient in need thereof a therapeutically effective amount of an anti-selectin agent, wherein said treatment reduces pathogen load in the lung.

2. The method of claim 1, wherein said agent specifically binds to E-selectin, L-selectin or P-selectin.

3. The method of claim 2, wherein said agent is an antibody, or antigen binding fragment thereof.

4. The method of claim 3, wherein said antibody, or antigen binding fragment thereof, specifically binds an antigenic determinant on E-selectin and/or L-selectin.

5. The method of claim 4, wherein said antibody specifically binds an antigenic determinant on E-selectin and an antigenic determinant on L-selectin, wherein said binding simultaneously or individually inhibits E-selectin and L-selectin functions.

6. The method of claim 5, wherein said antibody is effective in a dose range of 0.05 mg/kg to 5 mg/kg.

7. The method of claim 5, wherein said antibody is effective in a dose of about 1 mg/kg.

8. The method of claim 5, wherein said antibody is monoclonal antibody EL246 secreted by a hybridoma having ATCC Accession No. HB 11049.

9. The method of claim 5, wherein said antibody, or antigen binding fragment thereof, has the same binding specificity for either or both L-selectin or E-selectin as monoclonal antibody EL246.

10. The method of claim 5, wherein said antibody, or antigen binding fragment thereof, competes with monoclonal antibody EL246 for binding with either or both E-selectin or L-selectin.

11. The method of claim 5, wherein said antibody, or antigen binding fragment thereof, binds to the same epitope on L-selectin and/or E-selectin as monoclonal antibody EL246.

12. The method of claim 3, wherein said antibody is a human, humanized or chimeric antibody.

13. The method of claim 1, wherein said patient is an adult patient.

14. The method of claim 1, wherein said patient is a mammal.

15. The method of claim 14, wherein said mammal is a human.

16. The method of claim 1, wherein said infection is a bacterial, fungal or viral infection.

17. The method of claim 16, wherein said infection is bacterial.

18. The method of claim 17, wherein said bacteria is selected from the group consisting of Pseudomonas aeruginosa, Staphylococcus aureus, Haemphilus influenzae, Moraxella catarrhalis, Legionella pneumophila, Klebsiella pneumonia, Streptococcus pneumoniae, Chlamydia pneumoniae, Mycoplasma pneumoniae, Bacillus anthracis, and Burkholeria cepacia.

19. The method of claim 1, wherein said patient has been diagnosed with a disorder selected from the group consisting of chronic bronchitis, chronic obstructive pulmonary disease (COPD), pneumonia, pneumonitis, acute respiratory distress syndrome (ARDS), severe acute respiratory syndrome (SARS), sarcoidosis, cystic fibrosis, emphysema, asthma, Smoker's Cough, allergy, allergic rhinitis, sinusitis or pulmonary fibrosis.

20. The method of claim 1, wherein said agent is administered to said patient at a dosage of at least about 10 μg/kg.

21. The method of claim 1 wherein an anti-bacterial, anti-viral or anti-fungal drug is also administered to said subject.

22. The method of claim 1 wherein said patient is suffering from an acute exacerbation of chronic bronchitis, COPD or asthma.

23. A method of treating or preventing a pulmonary infection consisting essentially of administering to a patient in need thereof a therapeutically effective amount of an antibody, or antigen binding fragment thereof, which specifically binds an antigenic determinant on E-selectin and an antigenic determinant on L-selectin, wherein said binding simultaneously or individually inhibits E-selectin and L-selectin functions, and wherein said treatment reduces pathogen load in the lung.
